(19) United States
(12) Patent Application Publication
(10) Pub. No.: US 2011/0208066 A1
(43) Pub. Date: Aug. 25, 2011

(54) NONINVASIVE BLOOD PRESSURE MEASUREMENT AND MONITORING DEVICE

(76) Inventor: Alfred Peter Gnadinger, Colorado Springs, CO (US)

(21) Appl. No.: 12/710,178

(22) Filed: Feb. 22, 2010

(51) Int. Cl. A61B 5/021

(52) U.S. Cl. 600/485

(57) ABSTRACT

Measurement of blood pressure is one of the most common procedures done in a clinical and an ambulatory environment. It is usually done with a sphygmomanometer, where an inflatable cuff is attached to the arm of a patient and the systolic and diastolic pressures are determined, typically by listening to the Korotkoff sounds. Although this method is over 100 years old and widely used, it is well known that it has severe shortcomings.

The present invention covers a novel device and method to continuously measure blood pressure using a noninvasive approach. A surface acoustic wave (SAW) pressure sensor is placed on a flexible substrate and placed on the wrist of a patient. This blood pressure sensing device communicates wirelessly with a monitor that is placed several meters away. The monitor can also be a wristwatch worn by the patient. The invention further encompasses a calibration procedure to convert the relative blood pressure values into absolute values.

The main application for this novel device and method is in an intensive care environment where continuous monitoring of blood pressure on critically ill patients is important. Since the proposed method is inexpensive it can also be used by patients at home or even by healthy people (e.g. athletes). The reader system consists of an antenna and a standard computer (e.g. laptop) with signal processing software.

### TECHNICAL FIELD

The present invention relates to a noninvasive blood pressure measurement and monitoring device. Emphasis is placed on an inexpensive sensor device based on surface acoustic wave (SAW) technology that communicates with a remote computer system wirelessly.

### BACKGROUND AND PRIOR ART

Measurement of blood pressure is one of the most common procedures done in a clinical and an ambulatory environment. It is usually done with a sphygmomanometer, where an inflatable cuff is attached to the arm of a patient and the diastolic and systolic pressures are determined, typically by listening to the Korotkoff sounds. Instead of listening to the Korotkoff sounds with a stethoscope, automated systems are commercially available that determine the Korotkoff sounds with appropriate sensors. An example of such a system is the HEM-790IT, a health management system from OMRON Corporation. Although the sphygmomanometer is over 100 years old and widely used, it is well known that it has severe shortcomings. The main disadvantage as related to the present invention is that it does not allow for continuous monitoring of blood pressure, which is often required for severely ill patients in a hospital setting. At best, blood pressure can be measured every 5 minutes with a sphygmomanometer. In a hospital setting, quite often so called A-lines are used to measure arterial blood pressure. An A-line is an intra-vascular catheter, where the blood pressure is compared to the pressure of a liquid inside the catheter tubing. Since this is an invasive procedure it is only used when absolutely necessary. Furthermore, inserting a catheter into the arteries of a small child or severely ill patient with weak blood vessels is extremely difficult. Thus there is a need for a noninvasive, inexpensive and reliable way to measure and monitor blood pressure.

The continuous noninvasive measurement of blood pressure is known in the art. Examples are two patents by Eckerle (U.S. Pat. Nos. 4,802,488 and 4,269,193) where it is disclosed how intra-arterial blood pressure can be measured noninvasively by an electromechanical transducer that includes an array of transducer elements. The prior art, however, discloses expensive and cumbersome approaches, not well suited for typical applications.

### BRIEF SUMMARY OF THE INVENTION

This invention is based on detecting the continuous force signal generated by a blood vessel due to the overpressure inside the vascular system. The present invention uses a surface acoustic wave (SAW) sensor to detect these force variations. A SAW sensor typically responds to temperature and strain. The human body is an excellent thermostat so that we can assume that temperature is constant. It is then fairly straight forward to measure strain as a function of time. There is a direct correlation between the force variations, the strain in the SAW sensor and blood pressure so that blood pressure can be extracted from the raw sensor data.

The aim of the present invention is to provide an inexpensive blood pressure monitoring device that can be applied like a band-aid to the wrist of a patient and that communicates wirelessly with a remote computer system. The computer system is placed a certain distance, for example 2 to 10 meters, away from the patient. Instead of a band-aid the blood pressure monitoring device can take the form of a bracelet, part of the garment (front part of the sleeve) or any other form that applies a force on the wrist artery or arteries and stays in place when the person is moving around. The computer system can be any type commonly used. It can be a desktop or a laptop computer. It can also be a mobile device such as an iPhone® or a Blackberry®. A special embodiment of the present invention has the computer system reside in a wristwatch that is worn on top of or adjacent to the blood pressure monitoring device.

The blood pressure monitoring device is passive, i.e. it does not contain a power source (e.g. a battery). The power is supplied by the reader system (i.e. the computer system). The blood pressure monitoring device contains three elements only: (1) the SAW sensor itself, (2) a thin, flexible, dielectric (non-conductive) substrate and (3) an antenna embedded in or attached to the substrate. The blood pressure monitoring device is in any form where the SAW sensor exerts a force on the artery or arteries in the wrist of a patient, such as a standard band-aid, about 4 cm long and 1.5 cm wide. As mentioned above, other forms such as a bracelet, a part of the garment (e.g. sleeve) are feasible.

Since the force applied between the bad-aid and the wrist of the patient is not controlled, only relative blood pressure values are obtained and calibration is needed to obtain absolute values of blood pressure. The system could be calibrated using a standard sphygmomanometer. However, a sphygmomanometer, although fairly accurate, is cumbersome to use. A simple, but effective calibration method is desirable and is an integral part of the present invention. The blood pressure measured by the device of the present invention is dependent on the position of the wrist relative to the heart of the patient. By raising and lowering the arm the values for both the systolic and the diastolic pressure are changed. The effect of a vertical displacement is most pronounced for the diastolic blood pressure. For the systolic blood pressure raising or lowering the arm with the wrist monitor does not alter the values significantly, since the systolic blood pressure also depends on the elasticity of the artery walls, a quantity that is not controlled and changes with age of the patient. The calibration method which is part of the present invention does, therefore, use the change in diastolic blood pressure as a function of the vertical displacement of the sensor with respect to the heart. Since the difference in diastolic blood pressure as a function of the vertical position of the blood pressure sensor can be calculated from the hydrostatic equation and these pressure values are absolute, this information can be used to calculate the absolute values of the diastolic blood pressure. From the absolute value of the diastolic blood pressure and the calibration constant, the systolic blood pressure can be calculated. More detail on the calibration method is given in the detailed description of preferred embodiments below.

The present invention is most useful in a hospital setting (e.g. ICU) where continuous blood pressure data in critically ill patients is required. In this situation, changes in blood pressure are important. However, the invention is also useful in ambulatory settings. Since the blood pressure monitoring device is inexpensive and the reader system can be built with a standard computer system, the proposed technology can also be used by people in home care, i.e. people can measure their blood pressure at home without attendance of a health care professional.

Since the method of the present invention is noninvasive and the blood pressure monitoring device is inexpensive, it can also be used by healthy persons such as athletes. The system of the present invention allows such a person to measure blood pressure continuously.

### BRIEF DESCRIPTION OF THE DRAWINGS

The present invention and its advantages will be better understood when the written description provided herein is taken in conjunction with the drawings wherein:

FIG. 1 is a top view of a surface acoustic wave (SAW) sensor of the reflector type;

FIG. 2 is a cross-sectional view of a surface acoustic wave (SAW) sensor of the reflector type;

FIG. 3 is a top view of a blood pressure monitoring device in form of a band-aid, including a SAW sensor, a flexible, dielectric substrate and a transducer antenna;

FIG. 4 is a cross-sectional view of a pressure monitoring device in form of a band-aid, including a SAW sensor, a flexible, dielectric substrate and a transducer antenna;

FIG. 5 shows the method of measuring the deformation of a blood vessel wall,

FIG. 6 is a block diagram of a complete blood pressure measuring and monitoring system;

FIG. 7 is a top view of a blood pressure monitoring device in form of a band-aid with multiple SAW sensors and one common transponder antenna; and

FIG. 8 shows a typical blood pressure signal as measured by the blood pressure measuring and monitoring device.

FIG. 9 shows a flow diagram of an embodiment of the calibration method for the blood pressure monitoring device.

For the sake of clarity the figures do not necessarily show the correct dimensions, nor are the relations between the dimensions always in a true scale.

### DESCRIPTION OF PREFERRED EMBODIMENTS

When describing the details of the various embodiments of the present invention, it is understood that it is directed at persons having a thorough understanding of the technology involved. For background information on surface acoustic wave sensors please refer to the book: “Acoustic Wave Sensors: Theory, Design, & Physico-Chemical Applications” by D. S. Ballantine Jr., Robert M. White, S. J. Martin, and Antonio J. Ricco (1996).

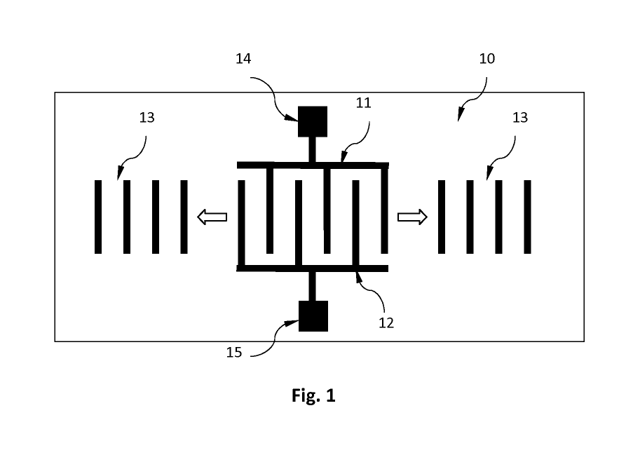

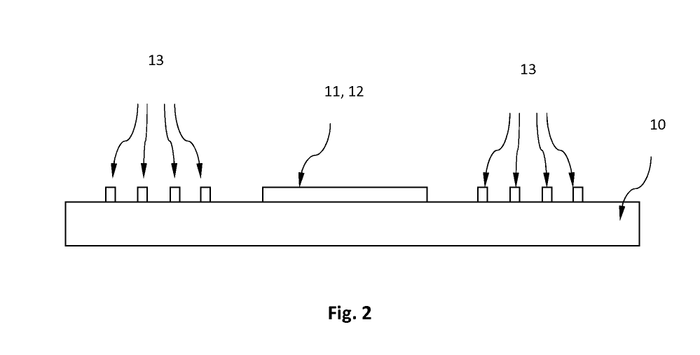

FIGS. 1 and 2 show the structure of a surface acoustic wave (SAW) sensor of the reflector type, whereby FIG. 2 is a cross section of FIG. 1.

A SAW sensor as used in the present invention and shown in FIGS. 1 and 2 consists of a piezoelectric substrate 10 and two interdigitated elements (IDT) 11 and 12. Reflectors 13 are provided on the same surface of the substrate 10 and at a certain distance from the ITD. The IDT is formed by depositing a conductive layer (e.g. aluminum) onto the surface of the piezoelectric substrate 10 and patterning it by using, for example, photolithography. Other patterning technologies are also feasible. For feature sizes greater than about 1.5μ, printing can also be used. The two combs of the IDT are connected to the outside world (e.g. the transponder antenna, not shown) with bond-pads 14 and 15. A preferred embodiment of the material for the piezoelectric substrate 10 is quartz (SiO₂). Other piezoelectric materials such as LiNbO₃, LiTaO₃, LiTiO₃, PZT, etc., can also be used. Although feasible are organic (plastic) piezoelectric materials such as polyvinylidene fluoride (PVDF). The main application for the present invention is as a blood pressure sensor. In this application the temperature is relatively low and well controlled and the above mentioned piezoelectric materials are well suited. For applications with a wider temperature range other materials are more appropriate. Examples are materials from the LGX family of crystals or gallium phosphate. ZnO is another example. SAW sensors are typically built on single crystal materials. However, polycrystalline materials are also feasible. They may show slightly degraded sensing properties but their low cost may offset the degraded performance. In the present invention, single crystalline piezoelectric materials are assumed.

Other types of SAW sensors, for example devices with two separate IDTs, one acting as a transmitter and one as a receiver of surface acoustic waves can be used. In the following description a reflector type SAW sensor is assumed.

If an ac voltage is applied to the two combs 11 and 12 of the IDT in FIGS. 1 and 2 through the bond-pads 14 and 15, a surface acoustic wave is generated that propagates along the surface of the device and is reflected by the reflectors 13. By choosing the correct frequency of the ac voltage the device can be operated in a resonant mode.

The propagation velocity of the surface acoustic wave is dependent on the physical parameters the device is subjected to, for example the stress/strain, the temperature, the chemical ambient, etc. If the surface acoustic wave sensor is in contact with the skin adjacent to a blood vessel, it can measure the pressure fluctuations of the vessel wall. SAW sensors are very sensitive because the propagating acoustic wave has its energy concentrated close to the device surface.

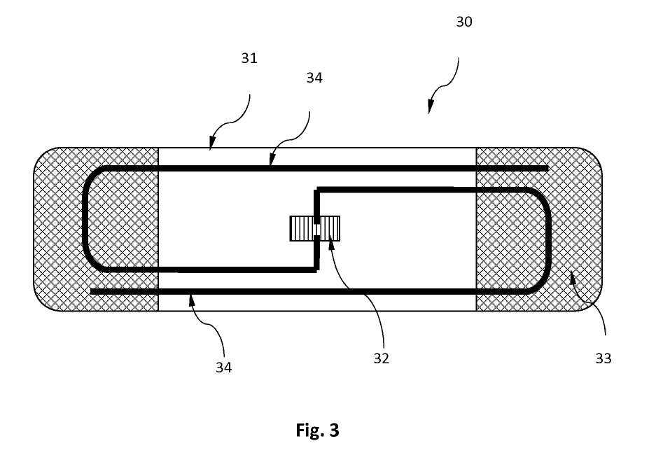

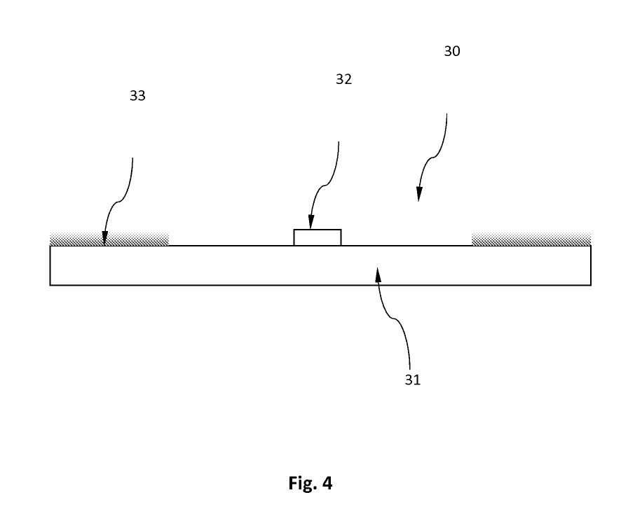

FIGS. 3 and 4 show the structure of one embodiment of the blood pressure monitoring device 30 of the present invention in the form of a band-aid, whereby FIG. 4 is a cross section of FIG. 3.

The blood pressure monitoring device 30 consists of a flexible, thin (typically less than 0.5 mm), dielectric substrate 31 as typically used for standard band-aids. Other configurations such as a bracelet, wrist band, sleeve of a garment, etc. could be used. The substrate 31 is, for example, 3 cm long and 1.5 cm wide. The two ends of the substrate are coated to a length of about 0.8 cm with an adhesive 33, as typically used in a band-aid. A surface acoustic wave sensor 32 is attached to the substrate 31 approximately in its center and on the same side as the adhesive 33. A transponder antenna 34 in the form of a microstrip antenna is provided on the substrate at it's periphery or is formed with wires that are attached to the substrate. The antenna can be printed onto the substrate, it can be electroplated or it can also be formed by other means, e.g. thin film deposition and photolithography. The dimensions of the antenna depend on the frequency employed for the communication scheme, but the width of the strip can always be large enough (e.g. 50μ) so that printing can be employed. The length of the antenna depends on the frequency. At 2.4 GHz, a possible communication frequency, the length of the antenna (λ/4) would be about 3 cm. For lower frequencies, the length of the antenna will be larger. Since the size of the substrate (band-aid) is given, the antenna can have multiple turns, if the optimal length is larger than the larger dimension of the substrate. The antenna can also be embedded into the (non-conductive) substrate. The antenna material can be any suitable conductor. Gold (Au) is a possible material choice. The thickness of the antenna trace is typically 20 to 50μ. One end each of the (dipole) antenna in FIG. 3 is connected to one bond pad (see FIG. 1) of the SAW sensor.

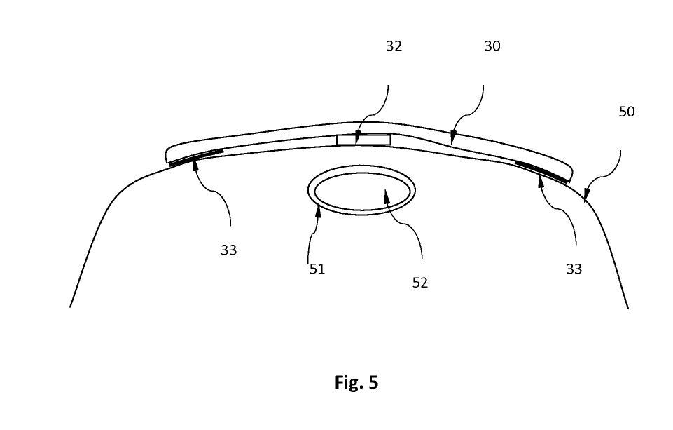

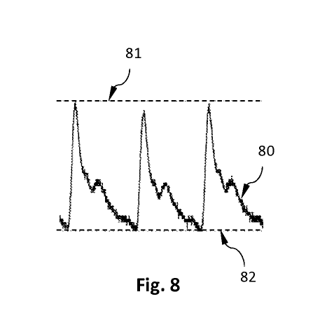

FIG. 5 shows how the blood pressure measuring system 30 is being used. The band-aid 30 is attached to the wrist of a person by means of the parts 33 with the adhesives with the SAW sensor touching the skin 50 of the wrist on top of an artery 52. The sensing device 32 deforms the blood vessel 52 slightly by deforming the surface of the wrist 50 in order to sense the movement of the blood vessel wall 51 vertical to the SAW sensor 32 in the sensing device 30. The SAW sensor measures a combination of physical parameters, for example temperature and strain. The temperature of the human body is well controlled so that the signal derived from the SAW sensor represents the strain experienced by the sensor under the influence of the pulsating artery. A typical strain signal as a function of time is shown in FIG. 8a. The force applied to the SAW sensor 32 in FIG. 5 is not controlled, so that the coordinates of the strain signal in FIG. 8a are relative. If absolute values of blood pressure are required, which is almost always the case, a calibration procedure has to be applied as will be described later.

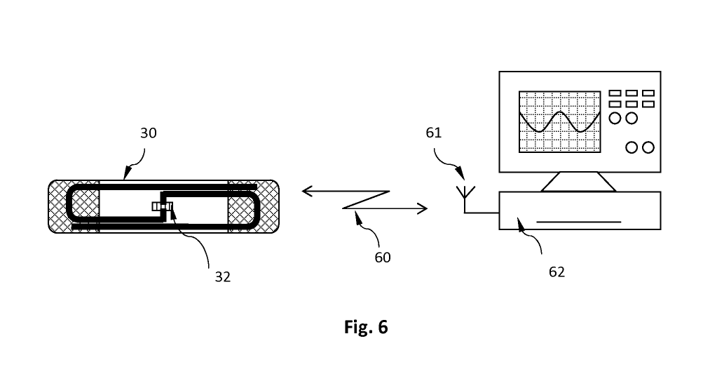

FIG. 6 shows a block diagram of an embodiment of the whole blood pressure measuring and monitoring system. The blood pressure measuring and monitoring system 30 as applied to the wrist of a person communicates with a computer system antenna 61 through a wireless communication channel 60. The antenna 61 is connected to the computer system 62 through a wire connection. The communication channel 60 can also be formed with wire connections, but in most cases this is impractical, except in special circumstances, for example in an ICU environment. The blood pressure measuring and monitoring system 30 transmits raw data to the computer system 62. All signal processing tasks are performed within the computer system 62. This keeps the costs of the blood pressure measuring and monitoring system 30 low. Power for the SAW sensor 32 (see FIG. 3) is provided by the computer system 62 through the connection 60 (wireless or with wires). The SAW sensors 32 are passive devices, that means that no power supply (e.g. batteries) are required for the SAW sensors 32.

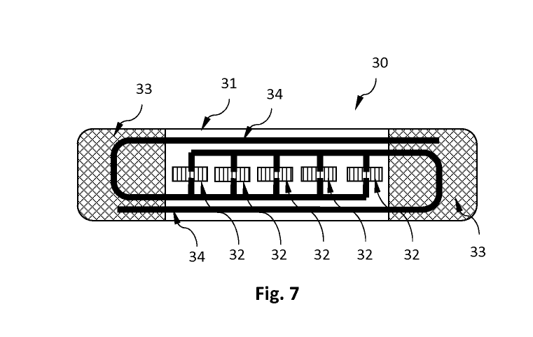

It may be difficult to locate the SAW sensors 32 exactly over a wrist artery or arteries where the signal would be largest. In another embodiment of the present invention, multiple SAW sensors 32 are placed in the central part of the band-aid substrate 31 as shown in FIG. 7. For typical dimensions of the SAW sensor chips 32 (for example, 3 mm×1 mm) and the band-aid 30 (4 cm×1.5 cm), about 5 sensors can be placed on a single band-aid substrate 31. These SAW sensors 32 can be connected to a common antenna 34 as shown in FIG. 7 or each SAW sensor 32 can have his own antenna. Anti-collision technology will be used to access one and only one SAW sensor 32. In this embodiment, the signals of the different SAW sensors are compared to each other and the one with the largest signal is chosen; the others are neglected. This is part of the signal processing task performed by the computer system 62 (FIG. 6). Since the SAW sensors are inexpensive, it is feasible to place redundant SAW sensors 32 on the band-aid substrate.

To be useful as a blood pressure measurement and monitoring device, the relative values of the systolic and diastolic blood pressure have to be converted into absolute values. That means that a calibration technique is required. One straight forward calibration scheme is using a sphygmomanometer to obtain absolute values of systolic and diastolic blood pressure, just before applying the blood pressure monitor of the present invention and relate them to the relative values of the blood pressure monitoring device. However, this calibration method is cumbersome and prone to errors. Calibration has to be performed periodically (e.g. every 10 minutes or so, depending on application) since the force exerted by the blood vessel on the SAW sensor on the band-aid diminishes with time, which makes using a sphygmomanometer even more cumbersome.

Since absolute values of blood pressure are only accurate within about 5%, a simple calibration method can be used that is part of the present invention. This calibration method is based on the hydrostatic pressure changes influencing the blood pressure values if the person lowers or raises the arm with the blood pressure monitoring device attached to the wrist. FIG. 8 shows a typical blood pressure signal 80 as measured by the blood pressure measuring and monitoring device 30 and transmitted to the computer system 62 (FIG. 6). This signal, with relative coordinates, is recorded and stored in the computer system 62. Of special interest are the maximum values 81 (systolic blood pressure) and the minimum values 82 (diastolic blood pressure).

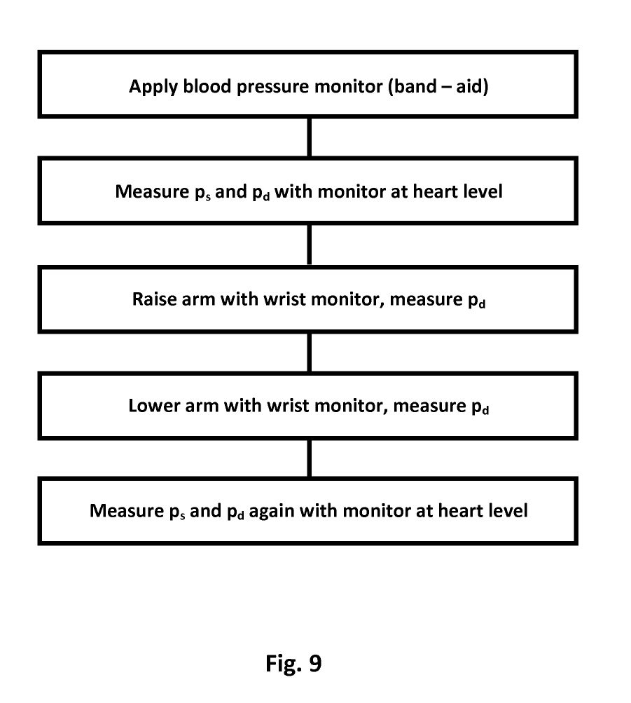

FIG. 9 shows a flow diagram of the calibration method. After applying the blood pressure monitor (band-aid) to one wrist of the patient, systolic blood pressure (pₛ) and diastolic blood pressure (p_{d}) are measured and transmitted wirelessly to the computer system where these values are stored. pₛ and p_{d} have relative coordinates. The patient now raises his/her arm and p_{d} is measured again. The calibration method does not use pₛ. It only uses p_{d}. pₛ is more variable since it depends among other parameters on the elasticity of the blood vessel, a quantity that is not controlled and changes with the age of the patient. With the arm with the pressure sensor raised, p_{d} is lower. The difference between p_{d} measured at heart level and p_{d} measured with arm raised is due to the hydrostatic pressure given by:

pₕ=ρ×g×h (1)

where pₕ is the hydrostatic pressure, ρ is the density of blood (approximately 1 g/cm³), g is the gravitational acceleration, g=9.8 ms⁻² at sea level and h is the vertical distance from the wrist sensor to the heart of the patient. Since the hydrostatic pressure pₕ is equal to the measured differences of the diastolic blood pressure with the sensor at heart level and the arm raised, h can be calculated from equation (1). To improve accuracy, the patient can lower the arm and the diastolic blood pressure can be measured again. Using again equation (1), h can be calculated. Assuming that the vertical distance between the heart and the raised wrist is about equal to the distance between the heart and the lowered wrist, the two values of h should be close. An average value can, therefore, be taken to improve accuracy.
The different values of p_{d} are, again, transmitted wirelessly to the computer system where they are stored.

We now have two different equations for the change in diastolic blood pressure p_{d}, one is equation (1) and the second one is given by:

Δp=p_{d}(arm raised)−p_{d}(heart level) (2)

Equation (1) is in absolute coordinates while equation (2) is in relative coordinates, related to the absolute values by the following equation

Δp=pₕ*x (3)

where x is a scaling factor.

We can now calculate x from (3), using (1) and (2).

After the calibration values are determined and transmitted to the computer system, pₛ and p_{d} are measured again with the wrist monitor at heart level. This time the computer will display the absolute values. All calculations are done in the computer system transparent to the patient and caregiver. As mentioned above, the calibration procedure has to be repeated periodically since the force applied to the wrist sensor may change with time.

The present invention has been described by way of a few examples, but this should not limit the scope of the protection since it is obvious to someone skilled in the art that the invention can easily be adopted to match different requirements in the field of blood pressure measurement and monitoring.

1. A noninvasive blood pressure measuring and monitoring device for continuously measuring and monitoring blood pressure in a blood vessel of a wrist of a person, comprising:
a. a dielectric, flexible substrate,
b. an antenna formed upon said dielectric, flexible substrate; and
c. at least one surface acoustic wave sensor attached to said dielectric, flexible substrate and electrically connected to said antenna,
whereby said pressure sensor senses the pulsation of said wrist artery of said person and said pulsation data is transmitted wirelessly to a reader system.

2. The blood pressure measuring and monitoring device according to claim 1, wherein the material of said surface acoustic wave sensor is quartz.

3. The blood pressure measuring and monitoring device according to claim 1, wherein the material of said surface acoustic wave sensor is lithium niobate.

4. The blood pressure measuring and monitoring device according to claim 1, wherein the material of said surface acoustic wave sensor is lithium tantalite.

5. The blood pressure measuring and monitoring device according to claim 1, wherein the antenna is embedded in said dielectric, flexible substrate and connected to said surface acoustic wave sensor by electrically conductive wires.

6. The blood pressure measuring and monitoring device according to claim 1, wherein said dielectric, flexible substrate is in the form of a band-aid.

7. The blood pressure measuring and monitoring device according to claim 6, wherein portions of said band aid are covered with an adhesive substance.

8. The blood pressure measuring and monitoring device according to claim 1, wherein a multitude of surface acoustic wave sensors are placed on said dielectric, flexible substrate and electrically connected to said antenna by electrically conductive wires.

9. The blood pressure measuring and monitoring device according to claim 1, wherein said surface acoustic wave sensor is calibrated to absolute values of blood pressure by raising and lowering the arm of the patient on whose wrist said blood pressure measuring and monitoring device is attached.

10. The blood pressure measuring and monitoring device according to claim 1, wherein said surface acoustic wave sensor is calibrated to absolute values of blood pressure by a standard cuff based blood pressure monitoring apparatus.

11. A method for making a blood pressure measuring and monitoring device for continuously and noninvasively measuring and monitoring blood pressure in a blood vessel, the method including the steps of:
a. Fabricating a surface acoustic wave sensor
b. Fabricating a transducer antenna and embedding said antenna in a dielectric, flexible substrate.

12. A method for using a blood pressure measuring and monitoring device according to claim 1 for continuously and noninvasively measuring and monitoring blood pressure in a blood vessel, the method comprising the steps of:
a. Extracting characteristic stress and strain data from said blood vessel,
b. converting said data into blood pressure data, and
c. transmitting said blood pressure data wirelessly to a remote computer system.

13. A method for using a blood pressure measuring and monitoring device according to claim 12, further comprising the step of calibrating the device in absolute pressure values by raising and lowering the arm of the patient on whose wrist said blood pressure measuring and monitoring device is attached.
